(19) United States
(12) Patent Application Publication
(10) Pub. No.: US 2013/0190890 A1
(43) Pub. Date: Jul. 25, 2013

(54) COMPOSITION AND METHODS FOR COATING

(71) Applicant: Massachusetts Institute of Technology, Cambridge, MA (US)

(72) Inventors: Nisarg J. Shah, Cambridge, MA (US); Jinkee Hong, Cambridge, MA (US); Nasim Hyder, Somerville, MA (US); Paula T. Hammond, Newton, MA (US)

(73) Assignee: MASSACHUSETTS INSTITUTE OF TECHNOLOGY, Cambridge, MA (US)

(21) Appl. No.: 13/746,902

(22) Filed: Jan. 22, 2013

Related U.S. Application Data
(60) Provisional application No. 61/589,179, filed on Jan. 20, 2012.

(51) Int. Cl. A61F 2/30

(52) U.S. Cl. CPC A61F 2/30
USPC 623/23.56; 623/18.11

(57) ABSTRACT

The present invention provides, among other things, multilayer film coating compositions, coated substrates and methods thereof. In some embodiments, a structure includes a first and second layer-by-layer film disposes on a substrate, the structure being characterized in that layer-by-layer removal of at least the second film releases at least one polypeptide, and also may permit release of ions from the ceramic material so that a synergetic effect of the osteoinduction and osteoconduction of the structure is achieved.

### RELATED APPLICATIONS

This application claims priority to U.S. provisional patent application Ser. No. 61/589,179, filed Jan. 20, 2012, the entire contents of which are herein incorporated by reference.

### GOVERNMENT SUPPORT

This invention was made with government support under Grant No. R01 AG029601 awarded by the National Institutes of Health. The government has certain rights in the invention.

### BACKGROUND

A major issue for the success of structural bone implants is failure due to aseptic loosening and non-optimal integration. The issue of establishing a stable, permanent bond between implant and parent bone is key in multiple applications, from one-stage dental implants to hip and knee and other whole joint replacement implants including revision surgery in which the implant is replaced in an environment where bone defects caused by bone lysis are usually present which can compromise stability of the new implant. The principal issues leading to failure in the above settings are the nature and integrity of the bond between the implant and the bone, the rate at which the bond forms and the amount of bone surrounding the implant that participates in stabilizing the device.

Although widely accepted as the technique of choice for cemented hip and knee replacement implants, self-curing poly(methyl methacrylate) (PMMA) based bone cements do not readily facilitate the formation of a reliable and mechanically coupled implant-bone interface due to a substantial elastic modulus mismatch at the bone interface. Further, PMMA is of low strength, not resorbable, and prone to fragmentation. The in situ formation of PMMA is a highly exothermic process that can cause local tissue necrosis, making it unfavorable for the incorporation and release of biologics that mediate the interaction between the host and implant. Thus, there is a continuing need for new insights on improved implant coatings and technologies.

### SUMMARY

The present invention provides, among other things, compositions, structures comprising such compositions and methods relating to such compositions.

In some aspects, the present invention provides compositions for coating a substrate (e.g., tissue, bone, scaffolds, permanent/resorbable implants and prosthetics), which compositions comprise one or more films. In some embodiments, a provided composition comprises a first film, wherein the film includes at least one bilayer comprising two polyelectrolyte layers of opposite charges, and wherein at least one polyelectrolyte layer comprises a ceramic material associated with the polyelectrolyte. In some embodiments, a provided composition comprises a second film. For example, in some embodiments, such a second film includes at least one tetralayer that releases an active agent such as a polypeptide.

In some embodiments, a provided composition comprises one or more films, which films include osteoconductive and osteoinductive agents. In some embodiments, a provided composition comprises a bilayer film and a tetralayer film; in some such embodiments, the bilayer film includes an osteoconductive agent (e.g., a ceramic material) and/or the tetralayer film includes an osteoinductive agent (e.g., a polypeptide, for example a bone growth factor). Without being bound by any particular theory, the present invention proposes that certain provided compositions achieve synergies between provided osteoconductive and osteoinductive agents (e.g., between an osteoconductive ceramic material and an osteoinductive polypeptide such as a bone growth factor). According to the present invention, in some embodiments, degradation and/or removal of one or more layers and/or one or more films achieves release of osteoconductive and/or osteoinductive agents. In some such embodiments, such release occurs so that synergy between activities of the osteoconductive and osteoinductive agents is observed. That is, in some embodiments, provided compositions achieve both osteoinduction and osteoconduction. In some embodiments, provided compositions may show synergies with respect to osteoinduction and/or osteoconduction.

In some embodiments, films utilized in accordance with the present invention are assembled by layer by layer deposition. In some embodiments, films utilized in accordance with the present invention degrade by layer by layer degradation.

In some aspects, the present invention provides layer-by-layer (LBL) films comprising a ceramic material complexed with a polyelectrolyte via non-covalent interactions (e.g. molecular ionic interactions). Among other things, the present disclosure provides the surprising insight and finding that LBL films described herein are particularly useful to stabilize complexed ceramic material(s) within films (e.g., via electrostatic interactions).

### DEFINITIONS

In order for the present disclosure to be more readily understood, certain terms are first defined below.

In this application, the use of “or” means “and/or” unless stated otherwise. As used in this application, the term “comprise” and variations of the term, such as “comprising” and “comprises,” have their understood meaning in the art of patent drafting and are inclusive rather than exclusive, for example, of additional additives, components, integers or steps. As used in this application, the terms “about” and “approximately” have their art-understood meanings; use of one vs the other does not necessarily imply different scope. Unless otherwise indicated, numerals used in this application, with or without a modifying term such as “about” or “approximately”, should be understood to cover normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of a stated reference value unless otherwise stated or otherwise evident from the context (except where such number would exceed 100% of a possible value).

“Associated”:

As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biodegradable”:

As used herein, the term “biodegradable” is used to refer to materials that, when introduced into cells, are broken down by cellular machinery (e.g., enzymatic degradation) or by hydrolysis into components that cells can either reuse or dispose of without significant toxic effect(s) on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis. In some embodiments, biodegradable polymeric materials break down into their component and/or into fragments thereof (e.g., into monomeric or submonomeric species). In some embodiments, breakdown of biodegradable materials (including, for example, biodegradable polymeric materials) includes hydrolysis of ester bonds. In some embodiments, breakdown of materials (including, for example, biodegradable polymeric materials) includes cleavage of urethane linkages.

“Hydrolytically Degradable”:

As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

“Nucleic Acid”:

The term “nucleic acid” as used herein, refers to a polymer of nucleotides. In some embodiments, nucleic acids are or contain deoxyribonucleic acids (DNA); in some embodiments, nucleic acids are or contain ribonucleic acids (RNA). In some embodiments, nucleic acids include naturally-occurring nucleotides (e.g., adenosine, thymidine, guanosine, cytidine, uridine, deoxyadenosine, deoxythymidine, deoxyguanosine, and deoxycytidine). Alternatively or additionally, in some embodiments, nucleic acids include non-naturally-occurring nucleotides including, but not limited to, nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups. In some embodiments, nucleic acids include phosphodiester backbone linkages; alternatively or additionally, in some embodiments, nucleic acids include one or more non-phosphodiester backbone linkages such as, for example, phosphorothioates and 5′-N-phosphoramidite linkages. In some embodiments, a nucleic acid is an oligonucleotide in that it is relatively short (e.g., less that about 5000, 4000, 3000, 2000, 1000, 900, 800, 700, 600, 500, 450, 400, 350, 300, 250, 200, 150, 100, 90, 80, 70, 60, 50, 45, 40, 35, 30, 25, 20, 15, 10 or fewer nucleotides in length)

“Physiological Conditions”:

The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

“Polyelectrolyte”:

The term “polyelectrolyte”, as used herein, refers to a polymer which under a particular set of conditions (e.g., physiological conditions) has a net positive or negative charge. In some embodiments, a polyelectrolyte is or comprises a polycation; in some embodiments, a polyelectrolyte is or comprises a polyanion. Polycations have a net positive charge and polyanions have a net negative charge. The net charge of a given polyelectrolyte may depend on the surrounding chemical conditions, e.g., on the pH.

“Polypeptide”:

The term “polypeptide” as used herein, refers to a string of at least three amino acids linked together by peptide bonds. In some embodiments, a polypeptide comprises naturally-occurring amino acids; alternatively or additionally, in some embodiments, a polypeptide comprises one or more non-natural amino acids (i.e., compounds that do not occur in nature but that can be incorporated into a polypeptide chain; see, for example, http://www.cco.caltech.edu/~dadgrp/Unnatstruct.gif, which displays structures of non-natural amino acids that have been successfully incorporated into functional ion channels) and/or amino acid analogs as are known in the art may alternatively be employed). In some embodiments, one or more of the amino acids in a protein may be modified, for example, by the addition of a chemical entity such as a carbohydrate group, a phosphate group, a farnesyl group, an isofarnesyl group, a fatty acid group, a linker for conjugation, functionalization, or other modification, etc.

“Polysaccharide”:

The term “polysaccharide” refers to a polymer of sugars. Typically, a polysaccharide comprises at least three sugars. In some embodiments, a polypeptide comprises natural sugars (e.g., glucose, fructose, galactose, mannose, arabinose, ribose, and xylose); alternatively or additionally, in some embodiments, a polypeptide comprises one or more non-natural amino acids (e.g., modified sugars such as 2′-fluororibose, 2′-deoxyribose, and hexose).

“Small Molecule”:

As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

“Substantially”:

As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Treating”:

As used herein, the term “treating” refers to partially or completely alleviating, ameliorating, relieving, inhibiting, preventing (for at least a period of time), delaying onset of, reducing severity of, reducing frequency of and/or reducing incidence of one or more symptoms or features of a particular disease, disorder, and/or condition. In some embodiments, treatment may be administered to a subject who does not exhibit symptoms, signs, or characteristics of a disease and/or exhibits only early symptoms, signs, and/or characteristics of the disease, for example for the purpose of decreasing the risk of developing pathology associated with the disease. In some embodiments, treatment may be administered after development of one or more symptoms, signs, and/or characteristics of the disease.

### BRIEF DESCRIPTION OF THE DRAWING

The Drawing, comprised of several Figures, is for illustration purposes only, not for limitation.

FIG. 1. According to certain embodiments of the invention, structured, multilayer coatings for bone regeneration are made up of two composite coatings. The base coating contains (a) chitosan (75-85% deacytelated chitin and (b) hydroxyapatite (HAP, Ca₁₀(PO₄)₆(OH)₂) with (c) poly(acrylic acid) (MW˜450 k) in a bilayer repeat unit. The osteogenic factor coating contains (d) a hydrolytically degradable poly(β-amino ester) and (e) rhBMP-2 that are alternated with poly(acrylic acid) on top of the osteoconductive base coating. (f) Schematics of the two sets of multilayers base coating and osteogenic coating demonstrate that the thickness of each can be tuned to make the surface more osteoconductive or increase the osteogenic drug load. (g) Release of rhBMP-2 can be tuned by varying the thickness of the degradable coating. The loading scales linearly with the number of tetralayers (R²=0.998, n=6). (h) rhBMP-2 loading has a dose dependent effect on the deposition of calcium, quantified by alizarin red at 7 days while the thickness of the osteoconductive base coating does not (n=6-9). A single factor ANOVA was used along with a Tukey post hoc test between different groups (s.d., **p<0.01; *p<0.05; ns=not significant; all others p<0.001). The HAP structure was rendered using available mineral data. The rhBMP-2 structure was extracted using NCBI's Cn3D MMDB program.

FIG. 2. In vivo evaluation of rhBMP-2 release and activity of exemplary compositions described herein was assayed by coating (a) smooth and drilled PEEK rods with different combinations of the coatings and (b) implanting the rods in the tibia of a rat. (c) Fluorescently labeled rhBMP-2 was tracked using an in vivo imaging system over approximately one month where (d) a decrease in the radiant efficiency at the implant site was observed over time (n=3-4). Bone marrow flushed out of excised tibiae was assayed for rhBMP-2 using ELISA where (e) a peak in the marrow accumulation was observed for smooth implants and (f) more sustained high levels of rhBMP-2 were observed for drilled implants, in both cases demonstrating dose-dependence (n=4-6). These observations indicate that the rhBMP-2 binds to the matrix at sites distal from the implant and may have a higher residence time in the implant pores for a sustained presence.

FIG. 3. Five color flow cytometry to identify mesenchymal stem cells differentiating into osteoblast cells for (a) control implants without a coating and exemplary implants described herein including coatings of (b) [Chi(HAP)/PAA]₂₀, (c) [Poly2/PAA/rhBMP-2/PAA]₆₀, (d) [Chi(HAP)/PAA]₂₀+[Poly2/PAA/rhBMP-2/PAA]₂₀ (e) [Chi(HAP)/PAA]₂₀+[Poly2/PAA/rhBMP-2/PAA]₄₀ and (f) [Chi(HAP)/PAA]₂₀+[Poly2/PAA/rhBMP-2/PAA]₆₀. (g) Quantitative results of the same FACS data shown with error as a function of time. There is an upregulation in osteoblast cells when a coating is present which is dose dependent at least 2 weeks post implantation suggesting that higher doses of BMP-2 may expedite early fixation. A synergistic effect is observed when both the osteoconductive base layer and rhBMP-2 is present. A single factor ANOVA was used along with a Tukey post hoc test between different groups (n=6 per time point, s.d., **p<0.01; *p<0.05; ns=not significant; all others p<0.001.). Cells that are CD29⁺CD44⁺CD45⁻CD90⁺BMPRI/II⁺ are MSCs differentiating into the osteoblast lineage.

FIG. 4. Pull-out tensile testing of exemplary implants described herein from the tibia. Implants within the tibia were subject to pull-out tensile tests and the data are presented from (a) non-porous and (b) porous implants. Pull out force increases linearly with time and dose effects are observed at early times up to 2 weeks post implantation, consistent with previous observations of the activated cell population (n=4-6 per time point). Bone formation on the implant surface is statistically distributed and forms connections with the native bone in (c) smooth and (d) drilled implants coated with both multilayers containing both HAP and rhBMP-2. As the number of connections increase, the tensile force needed to separate the implant increases. The larger surface area provided by the drilled holes increases the bone contact area and contributes to the further increase in pull-out force. A single factor ANOVA was used along with a Tukey post hoc test between different groups (s.d., **p<0.01; *p<0.05; ns=not significant; all others p<0.001.). Interfacial tensile strength data are provided in Supplementary Data Tables 1 and 2.

FIG. 5. Histology of implants with various coating formulations according to exemplary embodiments of the invention, demonstrating bone tissue morphogenesis at the implant interface. (a) to (f) are implants coated with [Chi(HAP)/PAA]₂₀+[Poly2/PAA/rhBMP-2/PAA]₄₀ at (a) & (d) 1 week, (b) & (e) 2 weeks, (c) & (f) 4 weeks. (g) [Chi(HAP)/PAA]₂₀+[Poly2/PAA/rhBMP-2/PAA]₆₀ at 1 week. The plane of fracture in implants with this coating can be visualized by comparing (g), (h) and (i) at 4 weeks which depict (g) an intact implant, (h) implant that has been partially separated and (i) implant entirely separated. In all these images, the bone-implant interface is intact, and cohesive failure occurs in the newly formed bone. (j) through (l) are sections of drilled implants subject to tensile testing after 4 weeks, which were coated with [Chi(HAP)/PAA]₂₀ and 20, 40, 60 tetralayers of [Poly2/PAA/rhBMP-2/PAA]. In all implants, maturing bone tissue is observed to adhere to the implant surface indicating that failure is due to fracture between new and existing bone, rather than the bone and implant. (m) Within the pores, the bone is observed to grow in a loop like conformation, where the outside matures (n) and gradually fills up the pore at 4 weeks (o). In all sections collagen deposition and cement lines with osteoblasts actively depositing bone are observed. Trichrome stains (a, d, f, g, j, k, m, n, o) and hematoxylin and eosin stains (b, c, e, h, i, l) are viewed under brightfield and (n) is viewed under polarized light. Scale bars in (a) and (c) are 200 μm, (b) is 50 μm (d-f, j-i) and (m-o) are 50 μm, (g-l) are 20 μm.

FIG. 6. Microcomputed tomography (μCT) imaging allows for qualitative and quantitative comparison of bone formation. (a) Radiographs of bone formation around implants coated with different combinations of osteophilic films. These images were used to quantify bone regeneration. Complete, conformal bone apposition is observed in columns 4 and 5 at 4 weeks. (b) through (i) Bone coverage and volume calculated at 4 weeks indicate significant improvements in bone regeneration and apposition when the complete osteogenic coating is present. Implants coated with the osteoconductive base coating and rhBMP-2 qualitatively demonstrated higher bone volume close to the bone-implant interface compared to animals treated with either one of the coatings individually or no coating. (f) through (i) Early calcification of the periosteum was observed in the presence of HAP which boosted calcification in the presence of rhBMP-2. A single factor ANOVA was used along with a Tukey post hoc test between different groups (n=4 per time point, s.d., **p<0.01; *p<0.05; ns=not significant; all others p<0.001.) depicts a top view of a writable-erasable product.

FIG. 7. Osteogenic layer-by-layer film characteristics. (a) Nanoindentation load vs. depth curves indicate that the addition of hydroxyapatite increases the elastic modulus of the films that is consistent with the elastic modulus of bone. (b) Loading of rhBMP-2 in LbL films has a linear (R²=0.998) correlation with respect to the number of drug layers. (c) Total thickness of the films depends on the number of layers in the film and (d) the osteoconductive base layer is observed to stay on the implant surface after release of rhBMP-2.

FIG. 8. Location of exemplary implant in the proximal tibia. Here, titanium pins have been used to provide contrast on a single image. The implant spans the width of the medullary canal. (a) The implant location as rendered by OsiriX® image processing software. (b) The same image rendered using GE Healthcare Microview® software that provides 3D visualization of the site.

FIG. 9. Birefringence of aligned collagen fibrils on an exemplary coating, [Chi(HAP)/PAA]₂₀+[Poly2/PAA/rhBMP-2/PAA]₆₀ at 4 weeks. Mature, aligned collagen fibrils in the new bone are observed in the periprosthetic space and the bone is conformal to the implant shape. Aligned collagen is also observed in the new bone at sites distant from the implant. Arrows indicate regions where significant birefringence is observed.

FIG. 10. Arrangement of new bone on [Chi(HAP)/PAA]₂₀ coated PEEK implants according to certain embodiments of the invention. There is a lack of new, mature collagen fibrils in the periprosthetic space at (a) 1 week (b) 2 weeks and (c) 4 weeks. (d) Aligned collagen is present at sites distant from the implant. (e) Regeneration of bone tissue is patchy within the implant pores. (f) The new bone does remain tethered to the implant pore wall after the pull-out test.

FIG. 11. Arrangement of new bone on [Poly2/PAA/rhBMP-2/PAA]₆₀ coated PEEK implants according to certain embodiments of the invention. (a, b, c) There is a lack of apposition of the new bone, which is located primarily in the periprosthetic space. (d, e) Aligned collagen is present at sites distal from the implant. (f) Regeneration of bone tissue within the implant pores is not conformal to the pore wall with few connections to the pore wall surface. However, the new bone does remain tethered to the implant pore wall after the pull-out test.

FIG. 12. Fibrotic tissue coating PEEK implants without a LbL coating described herein. (a, b) There is a lack of new bone formation in the periprosthetic space or the implant surface. There is a dominance of undifferentiated precursor cells and a thin layer of fibrous tissue around the implant. (c) The asterix indicates location of hematopoietic stem cells in the vicinity of the implant. (d, e) There is a lack of aligned collagen with osteocytes on or around the implant. (f) Regeneration of bone tissue within the implant pores is absent and the fibrous tissue detaches from the implant after the pull-out test, as above.

FIG. 13. NMR characterization of an exemplary degradable poly(β-amino ester) (Poly2). Representative ¹H NMR spectra of Poly-2 in CDCl₃. The methylene group at 4.03 ppm (t, 2H, —CH₂—OCO—CH₂—) has been taken as reference for proton integration.

FIG. 14. Average pull out tensile force with linear fit and 95% confidence intervals. Pull-out tensile force increases linearly for all coated implants with a coating (R²≧0.98). A comparison in the slopes indicated that for smooth implants, the differences in the slopes yields p<0.0001 and for drilled implants, the differences in the slopes yields p=0.003687.

### DETAILED DESCRIPTION OF CERTAIN EMBODIMENTS

In various embodiments, compositions, structures, and methods in accordance with the present invention are disclosed. In particular, compositions and methods for assembling LBL films associated with one or more agents are disclosed. Provided film compositions, structures, and methods can be used, for example, in the production and/or use of coated substrates, for example to achieve controlled loading and/or release of desired agents such as osteoconductive and/or osteoinductive agents.

### Layer-by-Layer (LBL) Films

LBL films may have any of a variety of film architectures (e.g., numbers of layers, thickness of individual layers, identity of materials within films, nature of surface chemistry, presence and/or degree of incorporated materials, etc), as appropriate to the design and application of coated devices as described herein.

In general, LBL films comprise multiple layers. In many embodiments, LBL films are comprised of multilayer units; each unit comprising individual layers. In some embodiments, adjacent layers are associated with one another via non-covalent interactions. Exemplary non-covalent interactions include, but are not limited to ionic interactions, hydrogen bonding interactions, affinity interactions, metal coordination, physical adsorption, host-guest interactions, hydrophobic interactions, pi stacking interactions, van der Waals interactions, magnetic interactions, dipole-dipole interactions and combinations thereof.

LBL films may be comprised of multilayer units in which alternating layers have opposite charges, such as alternating anionic and cationic layers. Alternatively or additionally, LBL films for use in accordance with the present invention may be comprised of (or include one or more) multilayer units in which adjacent layers are associated via non-electrostatic interactions.

According to the present disclosure, LBL films may be comprised of one or more multilayer units. In some embodiments, an LBL film may include multiple copies of a particular individual single unit (e.g., a of a particular bilayer, trilayer, tetralayer, etc unit). In some embodiments, an LBL film may include a plurality of different individual units (e.g., a plurality of distinct bilayer, trilayer, and/or tetralayer units). For example, in some embodiments, multilayer units included in an LBL film for use in accordance with the present invention may differ from one another in number of layers, materials included in layers (e.g., polymers, additives, etc), thickness of layers, modification of materials within layers, etc. In some embodiments, an LBL film utilized in accordance with the present invention is a composite that includes a plurality of bilayer units, a plurality of tetralayer units, or any combination thereof. In some particular embodiments, an LBL film is a composite that includes multiple copies of a particular bilayer unit and multiple copies of a particular tetralayer unit.

In some embodiments, LBL films utilized in accordance with the present invention include a number of a multilayer units that is about or at least a lower limit of 3, 4, 5, 6, 7, 8, 9, 10, 15, 20, 25 30, 35, 40, 45, 50, 55, 60, 65, 70, 75, 80, 85, 90, 95, 100, 150, 200, 300, 400 or even 500.

LBL films may have various thickness depending on methods of fabricating and applications. In some embodiments, an LBL film has an average thickness in a range of about 1 nm and about 100 μm. In some embodiments, an LBL film has an average thickness in a range of about 1 μm and about 50 μm. In some embodiments, an LBL film has an average thickness in a range of about 2 μm and about 5 μm. In some embodiments, the average thickness of an LBL film is or more than about 1 nm, about 100 nm, about 500 nm, about 1 μm, about 2 μm, about 3 μm, about 4 μm, about 5 μm, about 10 μm, bout 20 μm, about 50 μm, about 100 μm. In some embodiments, an LBL film has an average thickness in a range of any two values above.

Individual layers of LBL films can contain, be comprised of, or consist of one or more polymeric materials. In some embodiments, a polymer is degradable or non-degradable. In some embodiments, a polymer is natural or synthetic. In some embodiments, a polymer is a polyelectrolyte. In some embodiments, a polymer is a polypeptide.

LBL films can be decomposable. In many embodiments, LBL film layers are comprised of or consist of one or more degradable materials, such as degradable polymers and/or polyelectrolytes. In some embodiments, decomposition of LBL films is characterized by substantially sequential degradation of at least a portion of each layer that makes up an LBL film. Degradation may, for example, be at least partially hydrolytic, at least partially enzymatic, at least partially thermal, and/or at least partially photolytic. In some embodiments, materials included in degradable LBL films, and also their breakdown products, may be biocompatible, so that LBL films including them are amenable to use in vivo.

Degradable materials (e.g., degradable polymers and/or polyelectrolytes) useful in LBL films disclosed herein, include but are not limited to materials that are hydrolytically, enzymatically, thermally, and/or photolytically degradable, as well as materials that are or become degradable through application of pressure waves (e.g., ultrasonic waves).

Hydrolytically degradable polymers known in the art include for example, certain polyesters, polyanhydrides, polyorthoesters, polyphosphazenes, and polyphosphoesters. Biodegradable polymers known in the art, include, for example, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Those skilled in the art will recognize that this is an exemplary, not comprehensive, list of biodegradable polymers. Of course, co-polymers, mixtures, and adducts of these polymers may also be employed.

Anionic polyelectrolytes may be degradable polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself. Cationic polyelectrolytes may be degradable polymers with cationic groups distributed along the polymer backbone. Cationic groups, which may include protonated amine, quaternary ammonium or phosphonium-derived functions or other positively charged or ionizable groups, may be disposed in side groups pendant from the backbone, may be attached to the backbone directly, or can be incorporated in the backbone itself.

For example, a range of hydrolytically degradable amine-containing polyesters bearing cationic side chains have been developed. Examples of these polyesters include poly(L-lactide-co-L-lysine), poly(serine ester), poly(4-hydroxy-L-proline ester), and poly[α-(4-aminobutyl)-L-glycolic acid].

In addition, poly(β-amino ester)s, prepared from the conjugate addition of primary or secondary amines to diacrylates, are suitable for use. Typically, poly(β-amino ester)s have one or more tertiary amines in the backbone of the polymer, preferably one or two per repeating backbone unit. Alternatively, a co-polymer may be used in which one of the components is a poly(β-amino ester). Poly(β-amino ester)s are described in U.S. Pat. Nos. 6,998,115 and 7,427,394, entitled “Biodegradable poly(β-amino esters) and uses thereof” and Lynn et al., J. Am. Chem. Soc. 122:10761-10768, 2000, the entire contents of both of which are incorporated herein by reference.

In some embodiments, a polymer utilized in the production of LBL film(s) can have a formula below:

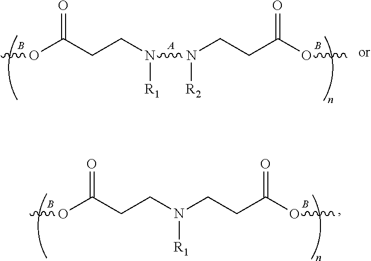

where A and B are linkers which may be any substituted or unsubstituted, branched or unbranched chain of carbon atoms or heteroatoms. The molecular weights of the polymers may range from 1000 g/mol to 20,000 g/mol, preferably from 5000 g/mol to 15,000 g/mol. In certain embodiments, B is an alkyl chain of one to twelve carbons atoms. In other embodiments, B is a heteroaliphatic chain containing a total of one to twelve carbon atoms and heteroatoms. The groups R₁ and R₂ may be any of a wide variety of substituents. In certain embodiments, R₁ and R₂ may contain primary amines, secondary amines, tertiary amines, hydroxyl groups, and alkoxy groups. In certain embodiments, the polymers are amine-terminated; and in other embodiments, the polymers are acrylated terminated. In some embodiments, the groups R₁ and/or R₂ form cyclic structures with the linker A.

Exemplary poly(β-amino esters) include

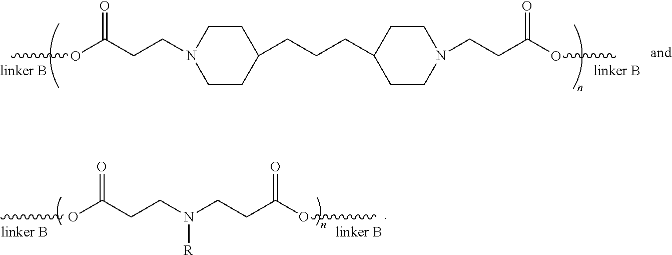

Exemplary R groups include hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups.

Exemplary linker groups B includes carbon chains of 1 to 30 carbon atoms, heteroatom-containing carbon chains of 1 to 30 atoms, and carbon chains and heteroatom-containing carbon chains with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups. The polymer may include, for example, between 5 and 10,000 repeat units.

In some embodiments, a poly(β-amino ester)s are selected from the group consisting of

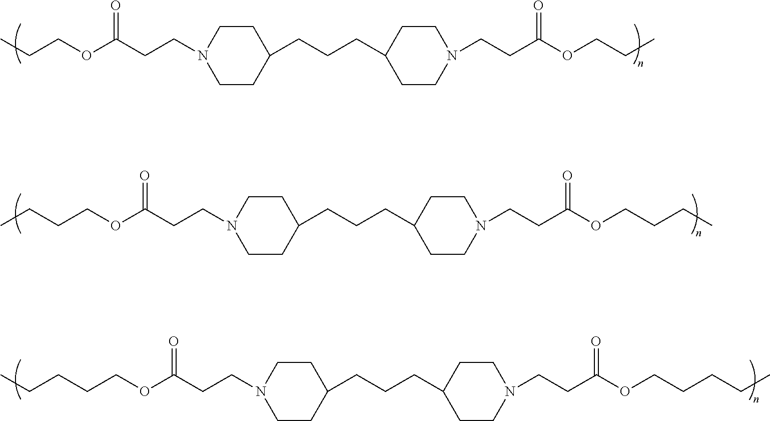

derivatives thereof, and combinations thereof.

Alternatively or additionally, zwitterionic polyelectrolytes may be used. Such polyelectrolytes may have both anionic and cationic groups incorporated into the backbone or covalently attached to the backbone as part of a pendant group. Such polymers may be neutrally charged at one pH, positively charged at another pH, and negatively charged at a third pH. For example, an LBL film may be constructed by LbL deposition using dip coating in solutions of a first pH at which one layer is anionic and a second layer is cationic. If such an LBL film is put into a solution having a second different pH, then the first layer may be rendered cationic while the second layer is rendered anionic, thereby changing the charges on those layers.

The composition of degradable polyelectrolyte layers can be fine-tuned to adjust the degradation rate of each layer within the film, which is believe to impact the release rate of drugs. For example, the degradation rate of hydrolytically degradable polyelectrolyte layers can be decreased by associating hydrophobic polymers such as hydrocarbons and lipids with one or more of the layers. Alternatively, polyelectrolyte layers may be rendered more hydrophilic to increase their hydrolytic degradation rate. In certain embodiments, the degradation rate of a given layer can be adjusted by including a mixture of polyelectrolytes that degrade at different rates or under different conditions.

In some embodiments, polyanionic and/or polycationic layers may include a non-degradable and/or slowly hydrolytically degradable polyelectrolytes. Any non-degradable polyelectrolyte can be used. Exemplary non-degradable polyelectrolytes that could be used in thin films include poly(styrene sulfonate) (SPS), poly(acrylic acid) (PAA), linear poly(ethylene imine) (LPEI), poly(diallyldimethyl ammonium chloride) (PDAC), and poly(allylamine hydrochloride) (PAH).

In some embodiments, the present invention utilizes polymers that are found in nature and/or represent structural variations or modifications of such polymers that are found in nature. In some embodiments, polymers are charged polysaccharides such as, for example sodium alginate, chitosan, agar, agarose, and carragenaan. In some embodiments, polysaccharides include glycosaminoglycans such as heparin, chondroitin, dermatan, hyaluronic acid, etc. Those of ordinary skill in the art will appreciate that terminology used to refer to particular glycosaminoglycans sometimes also is used to refer to a sulfate form of the glycosaminoglycan, e.g., heparin sulfate, chondroitin sulfate, etc. It is intended that such sulfate forms are included among a list of exemplary polymers used in accordance with the present invention.

In some embodiments, an LBL film comprises at least one layer that degrades and at least one layer that delaminates. In some embodiments, a layer that degrades in adjacent a layer that delaminates. In some embodiments, an LBL film comprises at least one polycationic layer that degrades and at least one polyanionic layer that delaminates sequentially; in some embodiments, an LBL film comprises at least one polyanionic layer that degrades and at least one polycationic layer that delaminates.

In some embodiments, one or more releasable agents is incorporated into one or more layers of an LBL film. In some embodiments layer materials and their degradation and/or delamination characteristics are selected to achieve a desired release profile for one or more agents incorporated within the film. In some embodiments, releasable agents are gradually, or other wise controllably, released from an LBL film.

In accordance with the present invention, LBL films may be exposed to a liquid medium (e.g., intracellular fluid, interstitial fluid, blood, intravitreal fluid, intraocular fluid, gastric fluids, etc.). In some embodiments, layers of the LBL films degrade and/or delaminate in such a liquid medium. In some embodiments, such degradation and/or delimination achieves delivery of one or more agents, for example according to a predetermined release profile.

### Agents

In some embodiments, the present invention provides compositions that comprise one or more agents. In some embodiments, agents may be released from LBL films. In some embodiments, an agent for delivery is released when one or more layers of a LBL film are decomposed and/or delaminated. Additionally or alternatively, in some embodiments, an agent or an ion of an agent may be released by diffusion.

In some embodiments, one or more agents are associated independently with a substrate, an LBL film coating the substrate, or both.

In some embodiments, an agent can be associated with one or more individual layers of an LBL film, affording the opportunity for exquisite control of loading and/or release from the film. In some embodiments, agents can be ceramic materials including bioceramics, bioglass, and metal oxide such as titanium oxide, iridium oxide, zirconium oxide, tantalum oxide, niobium oxide, cobalt oxide, chromium oxide. Exemplary bioceramics include, but are not limited to, hydroxyapatite (HAP), floroapatite, carbonate apatide, tricalcium phosphate, octacalcium phosphate, calcium pyrophosphate, tetracalcium phosphate, and dicalcium phosphate dehydrate. Exemplary bioglass includes, but are not limited to, SiO₂, Na₂O, CaO, and P₂O₅. For example, a model agent, HAP was complexed with a layer of polysaccharides as demonstrated in Examples below.

In some embodiments, an agent is incorporated into an LBL film by serving as a layer. For example, a polypeptide can serve as a layer and also as an agent for delivery. In some embodiments, a polypeptide is an osteogenic polypeptide and/or a growth factor. In some such embodiments, a polypeptide may be or comprise a bone morphogenetic protein (BMPs). For example, a model agent for delivery, BMP-2 served as a layer of a tetralayer as demonstrated in Examples below.

In theory, any agents including, for example, therapeutic agents (e.g. antibiotics, NSAIDs, glaucoma medications, angiogenesis inhibitors, neuroprotective agents), cytotoxic agents, diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be associated with the LBL film disclosed herein to be released.

In some embodiments, compositions and methods in accordance with the present disclosure are particularly useful for bone implants, prosthetics and/or scaffolds by incorporating one or more osteoconductive and/or osteoinductive agents. Exemplary osteoconductive agents include, but are not limited to, collagen-based scaffolds such as Healos (a polymer-ceramic composite consisting of collagen fibers coated with hydroxyapatite and indicated for spinal fusions); glass-based scaffolds; silicate-based scaffolds; ceramic-based substitutes; polymer-based substitutes, allografts; calcium phosphates such as hydroxyapatite, tricalcium phosphate, or fluorapatite; calcium sulfate; demineralized bone matrix; or any combination thereof. Additionally or alternatively, osteoconductive agents can be or may comprise fibronectin and/or collagen. Exemplary osteoinductive agents include, but are not limited to, BMPs, demineralized bone matrix, various growth factors known to be osteoinductive (e.g., transforming growth factor-α, growth and differentiation growth factor), stem cells or those with osteoblastic potential, etc. For example, growth factors can be selected from the group consisting of platelet-derived growth factor (PDGF), platelet-derived angiogenesis factor (PDAF), vascular endotheial growth factor (VEGF), platelet-derived epidermal growth factor (PDEGF), platelet factor 4 (PF-4), transforming growth factor beta (TGF-β), acidic fibroblast growth factor (FGF-α), basic fibroblast growth factor (FGF-β), transforming growth factor (TGF-α), insulin-like growth factors 1 and 2 (IGF-1 and IGF-2), B thromboglobulin-related proteins (BTG), thrombospondin (TSP), fibronectin, von Wallinbrand's factor (vWF), fibropeptide A, fibrinogen, albumin, plasminogen activator inhibitor 1 (PAI-1), osteonectin, regulated upon activation normal T cell expressed and presumably secreted (RANTES), gro-A, vitronectin, fibrin D-dimer, factor V, antithrombin III, immunoglobulin-G (IgG), immunoglobulin-M (IgM), immunoglobulin-A (IgA), a2-macroglobulin, angiogenin, Fg-D, elastase, keratinocyte growth factor (KGF), epidermal growth factor (EGF), fibroblast growth factor (FGF), tumor necrosis factor (TNF), fibroblast growth factor (FGF) and interleukin-1 (IL-1), Keratinocyte Growth Factor-2 (KGF-2), and combinations thereof.

Additionally or alternatively, compositions described herein include one or more therapeutic agents. Exemplary agents include, but are not limited to, small molecules (e.g. cytotoxic agents), nucleic acids (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof. In some embodiments, a therapeutic agent to be delivered is an agent useful in combating inflammation and/or infection.

In some embodiments, a therapeutic agent is or comprises a small molecule and/or organic compound with pharmaceutical activity. In some embodiments, a therapeutic agent is a clinically-used drug. In some embodiments, a therapeutic agent is or comprises an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, etc.

In some embodiments, a therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g., penicillin and clavulanic acid).

In some embodiments, a therapeutic agent may be an antibiotic. Exemplary antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof.

An antibiotic used in accordance with the present disclosure may be bacteriocidial or bacteriostatic. Other anti-microbial agents may also be used in accordance with the present disclosure. For example, anti-viral agents, anti-protazoal agents, anti-parasitic agents, etc. may be of use.

In some embodiments, a therapeutic agent may be or comprise an anti-inflammatory agent. Anti-inflammatory agents may include corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

Those skilled in the art will recognize that this is an exemplary, not comprehensive, list of agents that can be released using compositions and methods in accordance with the present disclosure. In addition to a therapeutic agent or alternatively, various other agents may be associated with a coated device in accordance with the present disclosure.

### Substrates

The present invention provides compositions comprising an LBL film, optionally including one or more agents, disposed upon a substrate. Any of a variety of materials or entities may be utilized as a substrate in accordance with the present invention.

In some embodiments, a substrate may be comprised of or may include a material such as a metals (e.g., gold, silver, platinum, and aluminum); a metal oxide, a coated metal, and combinations thereof.

In some embodiments, a substrate may be comprised of or may include a material such as a plastics, a ceramic, silicon, a glass, mica, graphite, and combinations thereof.

In some embodiments substrate may be comprised of or may include one or more polymers. Exemplary polymers for use as or in substrate materials include, but are not limited to, polyamides, polyphosphazenes, polypropylfumarates, polyethers, polyacetals, polycyanoacrylates, polyurethanes, polycarbonates, polyanhydrides, polyorthoesters, polyhydroxyacids, polyacrylates, ethylene vinyl acetate polymers and other cellulose acetates, polystyrenes, poly(vinyl chloride), poly(vinyl fluoride), poly(vinyl imidazole), poly(vinyl alcohol), poly(ethylene terephthalate), polyesters, polyureas, polypropylene, polymethacrylate, polyethylene, poly(ethylene oxide)s and chlorosulphonated polyolefins; and combinations thereof.

In some embodiments, a substrate may comprise more than one material (e.g., may be comprised of a composite material).

A substrate can be or comprise a medical device. Some embodiments of the present disclosure comprise various medical devices, such as sutures, bandages, clamps, valves, intracorporeal or extracorporeal devices (e.g., catheters), stents, vascular grafts, anastomotic devices, aneurysm repair devices, embolic devices, and implantable devices/scaffolds (e.g., orthopedic and dental implants) and the like. LBL films can be used in accordance with the present disclosure to coat such medical devices.

In some embodiments, a medical device is or comprises an orthopedic implant or prosthetic. Examples of orthopedic implants/prosthetics include without limitation total knee joints, total hip joints, ankle, elbow, wrist, and shoulder implants including those replacing or augmenting cartilage, long bone implants such as for fracture repair and external fixation of tibia, fibula, femur, radius, and ulna, spinal implants including fixation and fusion devices, maxillofacial implants including cranial bone fixation devices, artificial bone replacements, orthopedic cements and glues comprised of polymers, resins, metals, alloys, plastics and combinations thereof, nails, screws, plates, fixator devices, wires and pins and the like that are used in such implants, and other orthopedic implant structures as would be known to those of ordinary skill in the art. Alternatively or additionally, a medical device can be a scaffold used to replace and generate bone.

### Methods and Uses

There are several advantages to LBL assembly techniques used to coat a substrate in accordance with the present disclosure, including mild aqueous processing conditions (which may allow preservation of biomolecule function); nanometer-scale conformal coating of surfaces; and the flexibility to coat objects of any size, shape or surface chemistry, leading to versatility in design options. According to the present disclosure, one or more LBL films can be assembled and/or deposited on a substrate to provide a coated device. In many embodiments, a coated device having one or more agents for delivery associated with it, such that decomposition of layers of LBL films results in release of the agents.

In various embodiments, LBL films can be different in film materials (e.g., polymers), film architecture (e.g., bilayers, tetralayer, etc.), film thickness, and/or agent association depending on methods and/or uses. In many embodiments, compositions (e.g., a coated device) in accordance with the present disclosure are for medical use. In some embodiments, compositions and methods described herein are particularly useful for implants (e.g., orthopedic and dental implants).

It will be appreciated that an inherently charged surface of a substrate can facilitate LbL assembly of an LBL film on the substrate. In addition, a range of methods are known in the art that can be used to charge the surface of a substrate, including but not limited to plasma processing, corona processing, flame processing, and chemical processing, e.g., etching, micro-contact printing, and chemical modification.

In some embodiments, substrate can be coated with a base layer. Additionally or alternatively, substrates can be primed with specific polyelectrolyte bilayers such as, but not limited to, LPEI/SPS, PDAC/SPS, PAH/SPS, LPEI/PAA, PDAC/PAA, and PAH/PAA bilayers, that form readily on weakly charged surfaces and occasionally on neutral surfaces. Exemplary polymers can be used as a primer layer include poly(styrene sulfonate) and poly(acrylic acid) and a polymer selected from linear poly(ethylene imine), poly(diallyl dimethyl ammonium chloride), and poly(allylamine hydrochloride). It will be appreciated that primer layers provide a uniform surface layer for further LBL assembly and are therefore particularly well suited to applications that require the deposition of a uniform thin film on a substrate that includes a range of materials on its surface, e.g., an implant or a complex tissue engineering construct.

In some embodiments, assembly of an LBL film may involve a series of dip coating steps in which a substrate is dipped in alternating solutions. In some embodiments, LBL assembly of a film may involve mixing, washing or incubation steps to facilitate interactions of layers, in particular, for non-electrostatic interactions. Additionally or alternatively, it will be appreciated that LBL deposition may also be achieved by spray coating, dip coating, brush coating, roll coating, spin casting, or combinations of any of these techniques. In some embodiments, spray coating is performed under vacuum. In some embodiments, spray coating is performed under vacuum of about 10 psi, 20 psi, 50 psi, 100 psi, 200 psi or 500 psi. In some embodiments, spray coating is performed under vacuum in a range of any two values above.

Certain characteristics of a coated device may be modulated to achieve desired functionalities for different applications. Dose (e.g., loading capacity) may be modulated, for example, by changing the number of multilayer units that make up the film, the type of degradable polymers used, the type of polyelectrolytes used, and/or concentrations of solutions of agents used during construction of LBL films. Similarly, release kinetics (both rate of release and release timescale of an agent) may be modulated by changing any or a combination of the aforementioned factors.

In some embodiments, the total amount of agent released per square centimeter is about or greater than about 1 mg/cm². In some embodiments, the total amount of agent released per square centimeter in an LBL film is about or more than about 100 μg/cm². In some embodiments, the total amount of agent released per square centimeter in an LBL film is about or more than about 50 μg/cm². In some embodiments, the total amount of agent released per square centimeter in an LBL film is about or more than about 10 mg/cm², about 1 mg/cm², 500 μg/cm², about 200 μg/cm², about 100 μg/cm², about 50 μg/cm², about 40 μg/cm², about 30 μg/cm², about 20 μg/cm², about 10 μg/cm², about 5 μg/cm², or about 1 μg/cm². In some embodiments, the total amount of agent released per square centimeter in an LBL film is in a range of any two values above.

A release timescale (e.g., t_{50%}, t_{85%}, t_{99%}) of an agent for delivery can vary depending on applications. In some embodiments, a release timescale of an agent for delivery is less or more than about 1 hour, 2 hours, 3 hours, 4 hours, 5 hours, 10 hours, 15 hours, 20 hours, 25 hours, 30 hours, 40 hours, 50 hours, 75 hours, 100 hours, 150 hours, or 200 hours. In some embodiments, a release timescale of an agent for delivery is less or more than about 1 day, 2 days, about 5 days, about 10 days, about 12 days, about 20 days, about 30 days, 50 or about 100 days. In some embodiments, a release timescale of an agent for delivery is in a range of any two values above.

In some embodiments, provided compositions and/or structures (e.g., coated medical devices) are administered to (e.g., contacted with and/or implanted within) a subject in need thereof. In some such embodiments, the subject is suffering from or susceptible to one or more bone-related disorders. In some embodiments, the subject is undergoing or has undergone a surgical procedure.

### Exemplification

In this Example, the issue of establishing a stable, permanent bond between implant and parent bone is addressed by generating an ultrathin, conformal coating applicable to any implant or scaffold with an intricate geometrical surface that regulates release of tunable, physiologically relevant levels of BMP-2 while providing an osteoconductive matrix for direct adhesion of osteoblasts.

Materials:

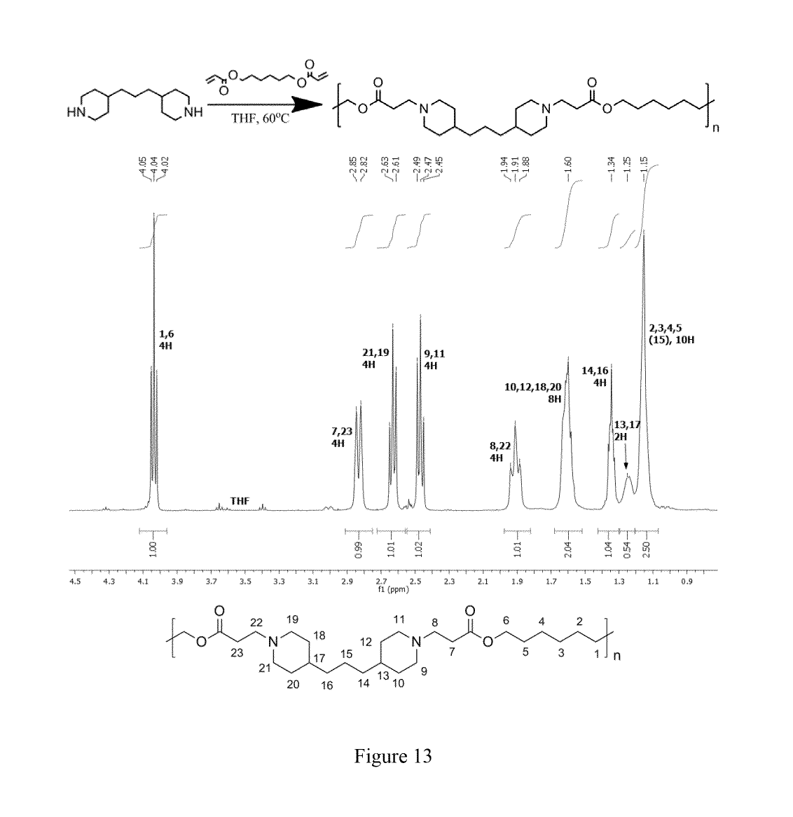

Poly 2, a poly(β-amino ester) (PBAE) was synthesized as previously described (Mₙ=11,910). The characterization and synthesis scheme are included (FIG. 13). All other materials were purchased from Sigma Aldrich (St. Louis, Mo.) or Invitrogen (Carlsbad, Calif.) unless otherwise noted.

Preparation of Electrostatic Layer-by-Layer (LBL) Films:

Hydroxyapatite (HAP) nanoparticle suspension (10 wt %) was diluted five-fold in sodium acetate buffer (0.1 M), sterile filtered through a 0.45 μm cellulose acetate membrane (VWR Scientific, Edison, N.J.) and added 1:1 (v/v) to chitosan solution in sodium acetate buffer (2 mg mL⁻¹). Poly(acrylic acid) (PAA) and Poly2 dipping solutions were prepared in sodium acetate buffer (1 mg mL⁻¹). One of the proven bone differentiation factors currently employed in the clinic is bone morphogenetic protein-2 (BMP-2). rhBMP-2 solution, a gift from Pfizer Inc. (Cambridge, Mass.), was diluted to 250 μg mL⁻¹ in sodium acetate buffer from a 10 mg mL⁻¹ stock solution. Poly(ether ether ketone) (PEEK) rods were purchased and machined into rods with diameter 1.3 mm and height 4 mm. Rods were drilled to make holes 150 μm in diameter. Flat PEEK substrates were machined from PEEK sheets with dimensions 10 mm×4 mm×1 mm (W_{X}L_{X}H). PEEK rods or sheets were plasma treated with air for 10 minutes and alternatively dipped into the prepared solutions with an intermediate washing step in water, first the osteoconductive [Chi(HAP)/PAA] base layers followed by the degradable [Poly2/PAA/rhBMP-2/PAA] layers.

Film Characterization:

Mechanical properties of the LbL films were determined using a Hysitron Triboindentor (Hysitron, Minneapolis, USA) with a conical tip (10 μm tip diameter) at a load of 1000 μN (ramp rate 25 μN/s) and dwell time of 3s was used. A standard fused quartz sample was used to calibrate the frame compliance, and diamond tip-area function needed to analyze the sample's hardness and elastic modulus. The indentations were performed at a depth of no more than 10% of the film thickness to avoid the effect of the underlying substrate. At least 16 (4×4 grid) indentations on each sample were performed from which the statistical average of the elastic modulus and hardness data were evaluated based on Oliver-Pharr method.

$$M = \frac{\sqrt{π}}{2} \cdot \frac{S}{\sqrt{A}} \qquad ( 1 )$$

where M is the indentation modulus of a homogeneous material, S is the contact stiffness, A is the projected area. Film thickness was measured using a Dektak 150 Profilometer. Films were scratched using a razor blade until the substrate was exposed. Film thickness was determined by the average step height of 3 scans 3000 μm long perpendicular to the scratch.

In Vitro Cellular Assays:

PEEK rods coated with rhBMP-2 were incubated in cell culture media (α-MEM supplemented with 20% FBS, 1% penicillin-streptomycin solution), which served as a release medium and incubated at 37° C. The release medium was periodically changed and assayed for rhBMP-2 using sandwich ELISA (Peprotech, N.J.). Adult mesenchymal stem cells (Lonza, Hopkinton, Mass.) were cultured in media as described above and seeded on PEEK sheets coated with the electrostatic films as described in 6 well tissue culture plates at 10,000 cells/well. Alkaline phosphatase and alizarin red assays were routinely performed using a previously described protocol.

In Vivo Animal Studies:

All animal work was performed in accordance with protocols approved by the Committee on Animal Care at the Massachusetts Institute of Technology. Adult male Sprague-Dawley rats (350-400 g) were used in this study. Animals were anesthetized and the right lower limb was shaved, disinfected with povidone iodine, and draped in a sterile manner. A 5 mm skin incision was made at the proximal tibial metaphysis in the region of the tibial tuberosity and extended to the underlying fascia and periosteum. The implant site was prepared by intermittent drilling a 1.4 mm unicortical hole through the cortical and cancellous bone below the patella ligament in order to gain access to the medullary cavity of the proximal metaphysis. This was done using a customized handheld drill (Aseptico, Wash.) with dental burrs (FST Inc., CA), and operated at a low rotary speed with saline irrigation. The implant (diameter 1.3 mm) was inserted without tapping and was flush with the external surface of the tibia entry site. The incision was closed in two layers with 5-0 polyglycolic acid sutures (Vicryl®, Somerville, N.J., USA) in subcutaneous tissues and skin, respectively. Animals were allowed unrestricted activity upon recovery and provided with analgesics. Live animal imaging was performed as described below and animals were sacrificed at pre-determined times.

In Vivo Tracking of Fluorescently Labeled rhBMP-2:

The IVIS® Spectrum pre-clinical imaging system (Caliper, Hopkinton, Mass.) was used to monitor fluorescently labeled rhBMP-2 at the implant site. rhBMP-2 was labeled with Alexa® 647 using the manufacturer's protocol and used to coat the implants as described above. Living Image software Version 3.0 (Caliper) was used to acquire and quantitate the fluorescence (excitation 640, emission 710). The implanted leg of the animal was shaved periodically to reduce background fluorescence. Under anesthesia, images were taken immediately after surgery and at most once every 24 hours after the first 4 weeks and every 48 hours subsequently until fluorescence was no longer detected.

Flow Cytometry Analysis:

After euthanasia, tibiae extending from the knee joint to the ankle were explanted. A 0.8 mm burr hole extending into the medullary cavity was drilled from the knee joint. A 22-gauge hypodermic needle was inserted into the medullary cavity and connected to a heparinized syringe. Bone marrow (1 to 1.5 ml) was aspirated while rotating and moving the needle back and forth. The medullary cavity was flushed with saline and the content aspirated. Samples were digested (45 min at 37° C.) with 0.25% trypsin in phosphate-buffered saline (PBS). Next, enzymes were inactivated with Dulbecco's modified Eagle's medium containing 10% fetal bovine serum, and recovered cells were washed with PBS. The phenotype of recruited cells was analyzed by flow cytometry with monoclonal antibodies against CD29, CD44, CD45, CD90, (BioLegend, CA) and BMPR1/R2 (Santa Cruz Biotechnology Inc., CA).

Histology and Pull Out Tensile Testing:

After euthanasia, tibiae were explanted and either fixed in 4% paraformaldehyde (PFA) or wrapped in PBS for immediate mechanical testing. PFA fixed samples were partially decalcified using a formic acid/hydrochloric acid mixture and embedded in glycol methacrylate, dehydrated, and stained with hematoxylin and eosin (H&E) and Masson's Trichrome stain to visualize various structural features. Pullout testing was performed to quantify implant mechanical fixation to surrounding bone tissue with an Instron® 5943 single column testing system. The ends of each excised tibia were secured, and the exposed head of the implant was connected to a load cell via a customized grip apparatus. Samples were then subjected to a constant pull rate of 0.1 N/s. The pullout force (N), parallel to the long axis of implant, was the maximum load achieved before implant detachment or failure. Interfacial shear strength was calculated by dividing the pull-out force by the total surface area of the implant.

Micro-Computed Tomography (μCT) Analysis:

Animals were anesthetized and imaged with μCT (eXplore CT120, GE Medical Systems, London, Ontario, Canada). The scanning protocol was performed with a 325 ms shutter speed, 2×2 binning, at X-ray tube parameters 70 kV and 50 mA. 220 images were taken at 0.877° incremental angles, with a gain of 100 and an offset of 20. Rendered images were reconstructed with the reconstruction Utility and analyzed using MicroView (GE Healthcare, Fairfield, Conn.). Threshold values were chosen by visual inspection and kept constant across all data sets and all time points. Regions of interest (ROIs) were chosen to cover the implant area, and the bone analysis tool was used to measure bone volume and bone coverage of each sample. Three dimensional representations of bone formation and two dimensional digital slices through the samples were also taken for qualitative comparison. Each sample was measured in triplicate for each set of data.

Statistical Analysis:

Results are presented as mean±standard deviation. Results were analyzed by analysis of variance (ANOVA) in Prism 5.04 (Graphpad Inc., CA). If deemed significant, pairwise comparisons were performed with Tukey post hoc test, and a confidence level of 95% was considered significant. In vitro assays were conducted in at least triplicate and replicated in three separate experiments.

### Interface of Functional Coating

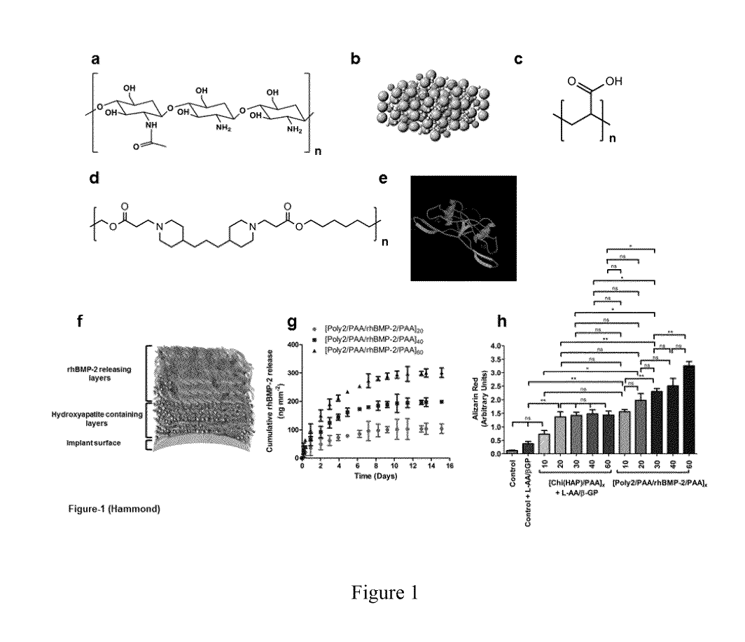

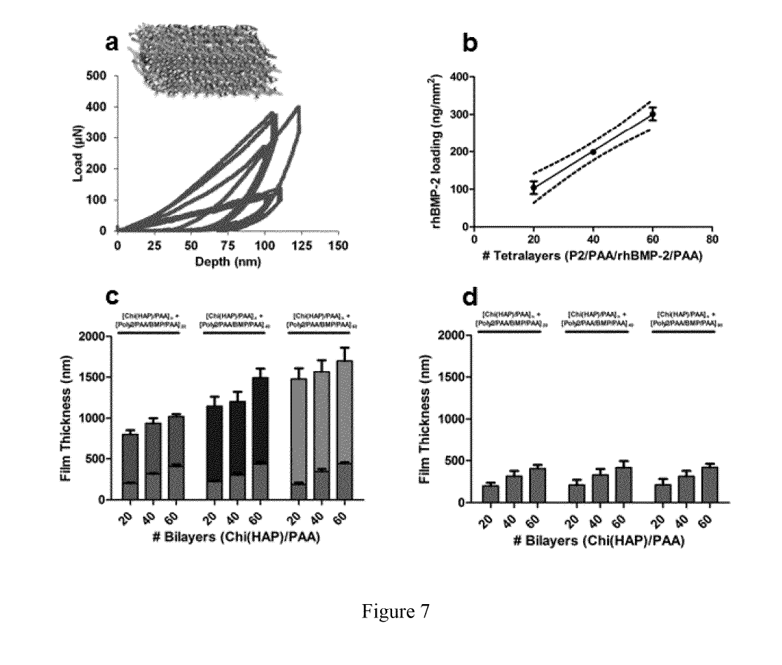

The LBL technique offers a unique platform for generating highly cohesive coatings with tunable biological and mechanical properties; we have used this method to develop osteogenic coatings with a composite architecture. The multicomponent film consisted of a set of osteoconductive base layers under a hydrolytically degradable multilayer film for promoting differentiation by introducing controlled amounts of osteoinductive rhBMP-2. This thin nanoblended film has an osteoconductive base coating, composed of HAP and cationic chitosan complexes, alternated with PAA (FIG. 1A-C). Among its many attributes, chitosan has is a linear cationic polysaccharide with minimal foreign body reaction and an intrinsic antibacterial nature. Nanoindentation of the osteoconductive base films revealed an elastic modulus of 14.52±0.85 GPa (FIG. 7A). This value is consistent with the elastic modulus of cortical bone and may be important to achieve the appropriate range of mechanical stiffness to support differentiation of osteoprogenitor cells that deposit trabecular bone on the implant surface for integration with the cortical bone. Implant materials have a range of elastic moduli which spans 3-100 GPa depending on the type of material. The LbL coating can lead to a graded transition between the implant substrate to the bone. The mechanical properties also suggest that the osteoconductive coating design would be able to withstand compressive stresses without fracturing. The elastic modulus dropped to 5.56±0.62 GPa in PAA/chitosan films without HAP, consistent with the observation that HAP plays a key role in the coating strength. The second component of the multilayer coating consisted of a degradable PBAE polycation that was alternated with PAA and rhBMP-2, to generate a set of layers atop this osteoconductive base multilayer (FIG. 1C-F). Under neutral to acidic pH conditions of film fabrication, this PBAE (Poly2) is stable and the amines present along the backbone of Poly2 are protonated, yielding a positive charge necessary for electrostatic LbL assembly. It is contemplated that films constructed with PAA as the alternating polyanion would yield ionically crosslinked films as this polymer exhibits a high charge density and would contribute to sustained release profile of rhBMP-2.

Controlled, Tunable Release with Surface Mediated Tissue Regeneration

Growth factor loading varies linearly with the number of layers as observed for degradable LbL coated implants with three different amounts of rhBMP-2, and a dose dependent effect was observed when released to osteoprogenitor human mesenchymal stem cells (FIG. 1I, FIG. 7). For control films without rhBMP-2, the rate and amount of calcium deposition by differentiating mesenchymal stem cells did not improve beyond 20 bilayers of the base coating in differentiation medium as measured by alizarin red staining for calcium deposits (FIG. 1I). The total rhBMP-2 dose for the in vitro studies varied from 4.1±0.7 μg at 20 tetralayers to 12.1±0.7 μg at 60 tetralayers of degradable growth factor film. At pH 7.4, the ester bond within the PBAE undergoes gradual hydrolysis, resulting in first order in vitro release of the rhBMP-2 in the LbL coating which lasted up to 2 weeks in cell culture media depending on the degree of BMP-2 loading—i.e. number of layers in the film (FIG. 1H). For all of the formulations, 90±1.1% of rhBMP-2 eluted after 1 week of release. The average rate of release per day in that time for all the drug loads was 12.7±0.1% of total load with no burst release was observed. This is important to ensure efficacy of the growth factor delivery system and is unlike known clinical bulk collagen carriers and depots in which 40-60% of the encapsulated protein is immediately released in the first three hours with low therapeutic effect. The growth factor release would not be tunable if chitosan was the only polycation used in the system because it would not be readily degradable; on the other hand, using Poly2 as the only polycation would result in the degradation of the entire multilayer structure, eliminating the ability to enhance bone growth with the permanent osteoconductive base layer.

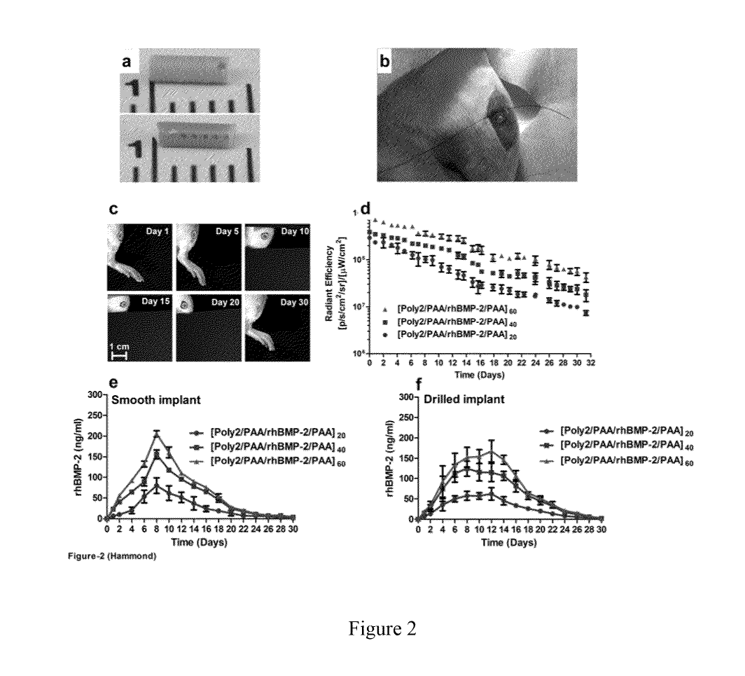

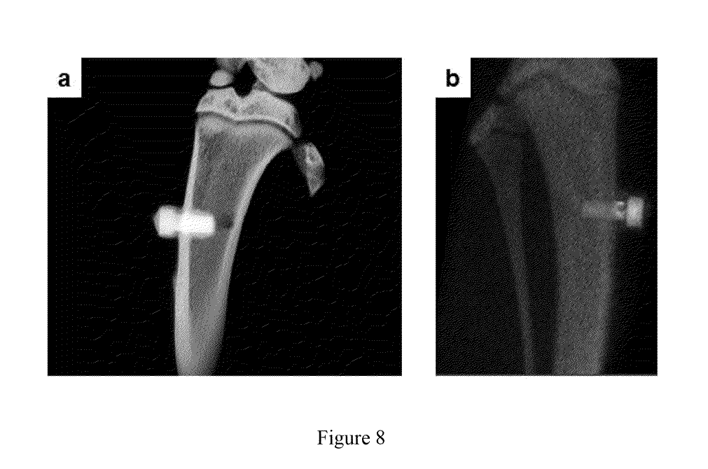

Implant integration and improvement of the rate and quality of tissue repair are the ultimate goals for biomaterial-based therapeutic strategies. As a test for such behavior, we examined the effects of our coating on PEEK implants. PEEK has radiolucent properties that permitted the use of radiography to monitor bone regeneration in real time compared to metal implants and correlate it with the intact implant embedded in the bone using histology. PEEK has also been increasingly used in orthopedic research as well as interbody spine fusion in the clinic. Smooth PEEK rods as well as rods containing 150 μm diameter holes drilled into the surface were investigated to enable evaluation of the role of implant geometry in these coating systems; in the clinic, porous ingrowth systems are generally designed to enable adhesion interlock with bone and increase surface area for bone integration. In our study, the PEEK was molded into rods and press-fit into circular unicortical defects drilled into the rat proximal tibia (FIG. 2A, B and FIG. 8). This model is a surrogate for dental and orthopedic clinical procedures in which the mechanical and biological integration of the implant with the surrounding bone is important to its stability.

The kinetics of rhBMP-2 release and dose of protein from degradable coatings can greatly influence bone regeneration and we anticipated that in vitro release studies only give a general idea of the actual release kinetics from implant surfaces in vivo. The effect of rhBMP-2 dosing on the formation and apposition of de novo trabecular bone, was evaluated using the three different multilayer BMP-2 loading formulations in an in vivo model. A near-IR fluorescent reporter was used to label rhBMP-2 and track its presence at the implant site over time in rodents (FIG. 2C, D). A gradual attenuation of the fluorescent signal from the implant site was observed for a period of approximately 1 month, at which time point the signal was no longer detectable at the lowest dose level over the background. These results are significantly different than the release timeframes observed in vitro, and the data suggest that available protein resides near the implant surface for days, rather than being rapidly cleared by the local vascular transport systems. Higher concentrations of rhBMP-2 were detectable by higher fluorescence as measured by specific radiant efficiencies. While the mode of drug release is primarily through hydrolytic degradation, diffusion may play a role, and the confined space within the bone defect leads to a much less significant sink than the highly dilute buffer solution used for in vitro measurements which, in combination with a slow clearance rate from the bone defect, can lead to slower release and an overall accumulation of BMP-2. Additional factors include the availability of BMP-2 binding sites in the neighboring tissue and extracellular matrix that may retain BMP-2, and differences in hydrolysis rate due to the adsorption of proteins and/or shifts in pH in the native wound healing environment of the defect space.

These observations were correlated with rhBMP-2 detected in homogenized bone marrow isolated from the tibia as well as FACS analysis for mesenchymal cells exhibiting differentiation into the osteoblast lineage. Based on ELISA measurements, rhBMP-2 was detected in the aspirates of animals with both drilled and smooth implants in a dose dependent manner. A peak in the dosage profile was observed several days after implantation of smooth PEEK implants followed by a subsequent monotonic reduction in rhBMP-2 (FIG. 2E); rhBMP-2 was detectable using ELISA for up to 4 weeks after implantation. It is conceivable that the released rhBMP-2 would proceed to bind to cell surface receptors or the bone matrix after release and the peak concentration observed for the smooth implant suggests a binding equilibrium for rhBMP-2, which is consistent with previous observations in vitro, with eventual dissociation and clearance of the protein. It is notable that the high in vivo localized concentration of BMP-2 at the implant site is maintained for multiple weeks. The observation of retention of rhBMP-2 is consistent with similar observations in other systems. In addition, the wounded tissue responds by forming a clot that rapidly stems the loss of blood and could limit the distance traveled by the released rhBMP-2 through the wound bed. Release from PEEK implants with drilled holes persisted over the same time scale as the smooth implants (FIG. 2F); however, we did not observe a defined peak in the rhBMP-2 accumulation with these porous implants, but a consistently high concentration over an extended multi-day period. This observation suggests that higher cumulative exposure to rhBMP-2 may be available at the implant site at later times, possibly due to reduced exposure to surrounding fluid and the sequestration of additional protein-loaded film within the holes. In vitro release studies and a mass balance of total drug load in vivo revealed that the porous implants had a 11±1.1% higher drug load, corresponding to the increase in the surface area due to the presence of the pores, indicating that the drug load scales with surface area due to the conformal nature of the coating. rhBMP-2 was not detected by ELISA in control animals without a coated implant. Attenuation of the fluorescent signal correlated with a drop in detectable levels of rhBMP-2 by ELISA, suggesting that it was due to the biologic being cleared from the body rather than a degradative loss of the fluorophore.

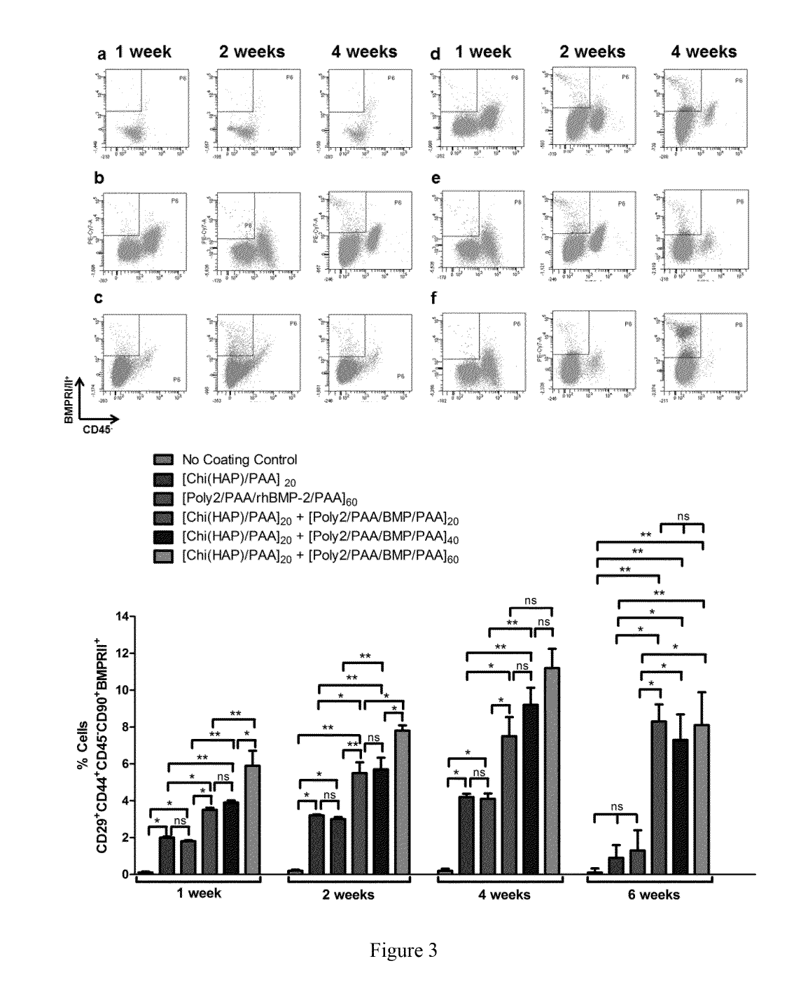

FACS analysis revealed an initial increase in the osteoblast cell population, which was dose dependent for the highest dose as detected by CD29⁺CD44⁺CD45⁻CD90⁺BMPR1/R2⁺ (FIG. 3). At 4 and 6 weeks, the activated cell population was indistinguishable within the rhBMP-2 dosed groups, suggesting that a higher dose is beneficial primarily for the initial upregulation of the osteoblast population and endogenous BMP production may increase osteoblast activity in the lower dosed groups. The peak activated osteoblast cell population decreases for the highest dose at week 6, which may signal the end of the bone deposition process and the beginning of homeostasis. The ability to reliably and consistently tune the dose of rhBMP-2 using the multilayer coating allows us to uniquely demonstrate and optimize the effect of rhBMP-2 dose on tissue morphogenesis both in vitro and in vivo.

Integration of the Implant with the Bone Tissue

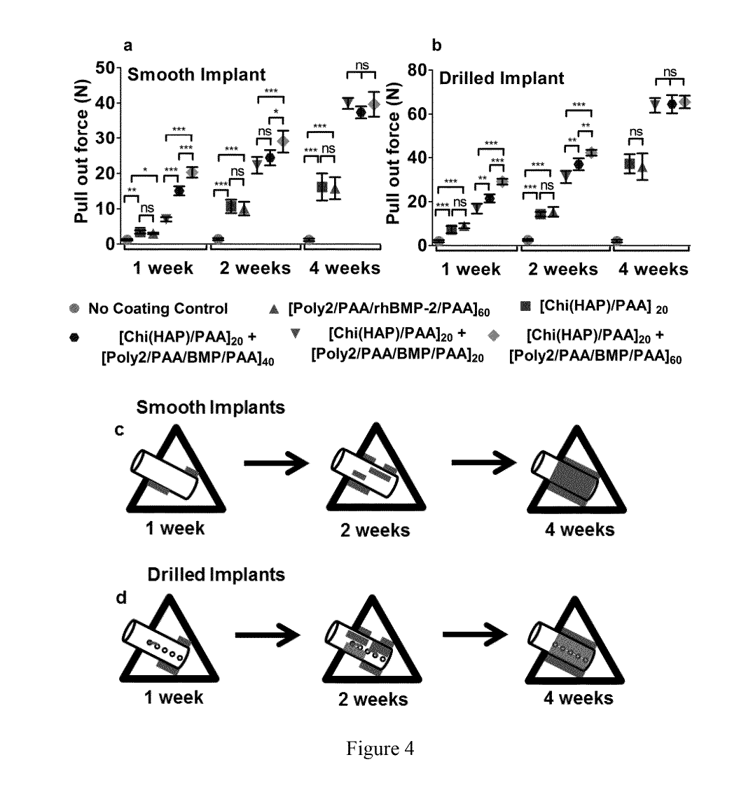

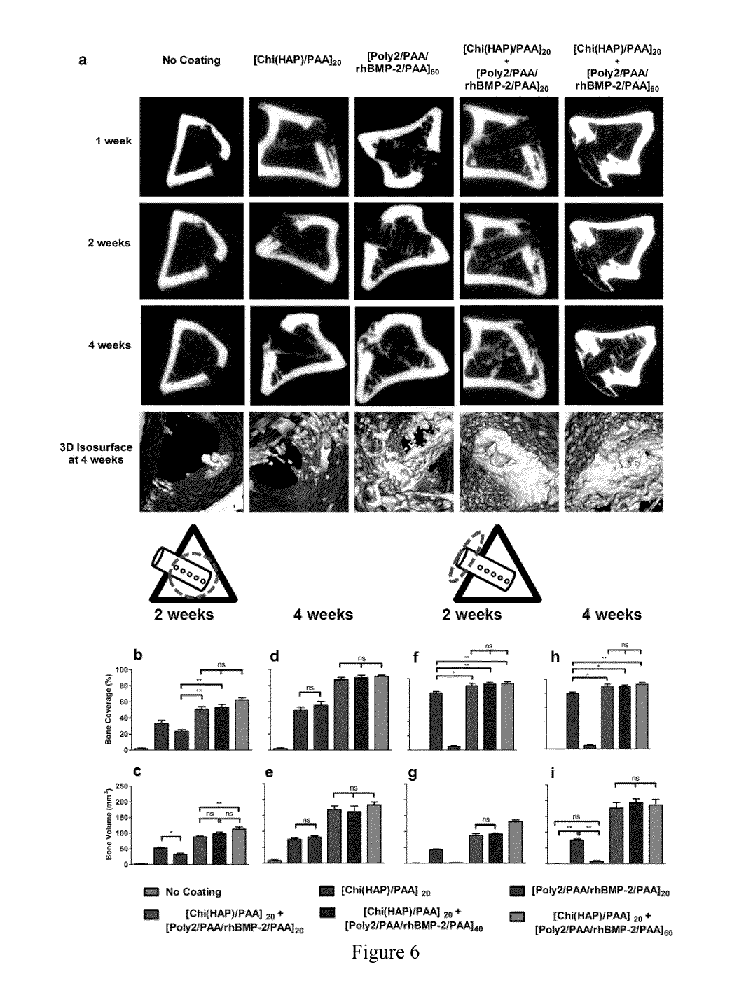

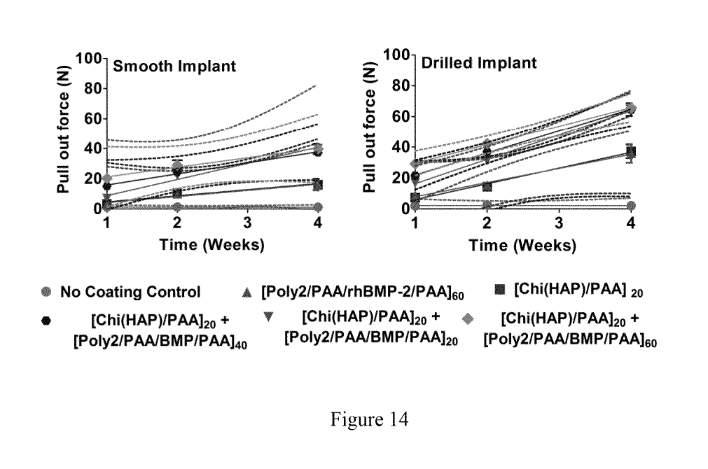

Mechanical tensile testing of the bone-implant interface was used to quantify the anchoring of the implant with bone, which provides a measure of the contribution of new bone to implant/bone osseointegration. In this setting, the interfacial tensile strength is derived by the bone adhesion to the implant surface and the connections that are made with the native bone tissue. The pull out force increased linearly over time in all groups with the LbL coatings (FIG. 4, FIG. 14). The introduction of rhBMP-2 and hydroxyapatite in the surface coating significantly increased the tensile force required to separate the implant from the bone when compared to uncoated implants or implants with either rhBMP-2 or HAP coatings separately (FIG. 4). The pull-out force for the implant with the highest dose of rhBMP-2 was significantly higher up to 2 weeks post-surgery, suggesting that a greater amount of trabecular bone is anchoring the implant with the parent bone, which is also consistent with previous observations of the activated osteoblast population (FIG. 4) and quantification of bone deposition from microCT (FIG. 6).

The contribution of hole size on tissue regrowth has been previously investigated^{37,38}. We investigated the effect of the coatings on ingrowth PEEK implants with drilled holes that were 150 μm in diameter. This hole size has been reported to encourage osteoid formation, and the ingrowth of mineralized bone. In these implants, additional bone formation within the holes was anticipated to provide additional resistance to tensile forces. The combination HAP and rhBMP-2 system provided a meaningful enhancement over either of the individual coating treatments. We observed a maximum interfacial tensile strength of 4.01±0.17 MPa following just 4 weeks for the drilled implants with the combination of HAP and rhBMP-2 LbL coatings (Table 1, 2). Interestingly, the pull-out force was found to be 32 times higher in the combination coating systems than uncoated implants at 4 weeks for both smooth and drilled implants. While the absolute values of interfacial tensile strength between the groups are different, the effect of geometry is accounted for by this normalization. In both groups, the maximum tensile force measured at 4 weeks was independent of rhBMP-2 dose. This trend was also observed in the FACS data and further suggests the role of endogenous BMP-2 in bone formation. The interfacial tensile strength of the combination coatings was found to be 2-3 times higher than HAP coatings on smooth implants using other methods of deposition and at least 3 times higher than bioactive bone cements. These data suggest that very early bonding between bioactive materials and living bone through the HAP layer is important to bone tissue apposition and tissue ingrowth.

TABLE 1
Interfacial tensile strength of non-porous implants calculated
from pull-out force data from FIG. 4. (values ± s.d.).

| | Interfacial Tensile Strength (MPa) | | |
|---|---|---|---|
| Group | 1 week | 2 weeks | 4 weeks |
| No Coating Control | 0.09 ± 0.02 | 0.10 ± 0.02 | 0.08 ± 0.03 |
| [Chi(HAP)/PAA]₂₀ | 0.24 ± 0.03 | 0.75 ± 0.13 | 1.13 ± 0.27 |
| [Poly2/PAA/rhBMP-2/PAA]₆₀ | 0.21 ± 0.02 | 0.70 ± 0.14 | 1.11 ± 0.22 |
| [Chi(HAP)/PAA]₂₀ + | 0.49 ± 0.04 | 1.67 ± 0.17 | 2.72 ± 0.13 |
| [Poly2/PAA/BMP/PAA]₂₀ | | | |
| [Chi(HAP)/PAA]₂₀ + | 1.05 ± 0.09 | 1.71 ± 0.15 | 2.62 ± 0.12 |
| [Poly2/PAA/BMP/PAA]₄₀ | | | |
| [Chi(HAP)/PAA]₂₀ + | 1.42 ± 0.10 | 2.17 ± 0.24 | 2.77 ± 0.24 |
| [Poly2/PAA/BMP/PAA]₆₀ | | | |

TABLE 2
Interfacial tensile strength of porous implants calculated
from pull-out force data from FIG. 4. (values ± s.d.).

| | Interfacial Tensile Strength (MPa) | | |
|---|---|---|---|
| Group | 1 week | 2 weeks | 4 weeks |
| No Coating Control | 0.14 ± 0.04 | 0.18 ± 0.03 | 0.14 ± 0.04 |
| [Chi(HAP)/PAA]₂₀ | 0.51 ± 0.11 | 1.00 ± 0.08 | 2.60 ± 0.30 |
| [Poly2/PAA/rhBMP-2/PAA]₆₀ | 0.63 ± 0.08 | 1.07 ± 0.16 | 2.51 ± 0.42 |
| [Chi(HAP)/PAA]₂₀ + | 1.18 ± 0.16 | 2.18 ± 0.20 | 4.47 ± 0.23 |
| [Poly2/PAA/BMP/PAA]₂₀ | | | |
| [Chi(HAP)/PAA]₂₀ + | 1.50 ± 0.13 | 2.58 ± 0.19 | 4.51 ± 0.29 |
| [Poly2/PAA/BMP/PAA]₄₀ | | | |
| [Chi(HAP)/PAA]₂₀ + | 2.03 ± 0.09 | 2.95 ± 0.09 | 4.58 ± 0.20 |
| [Poly2/PAA/BMP/PAA]₆₀ | | | |

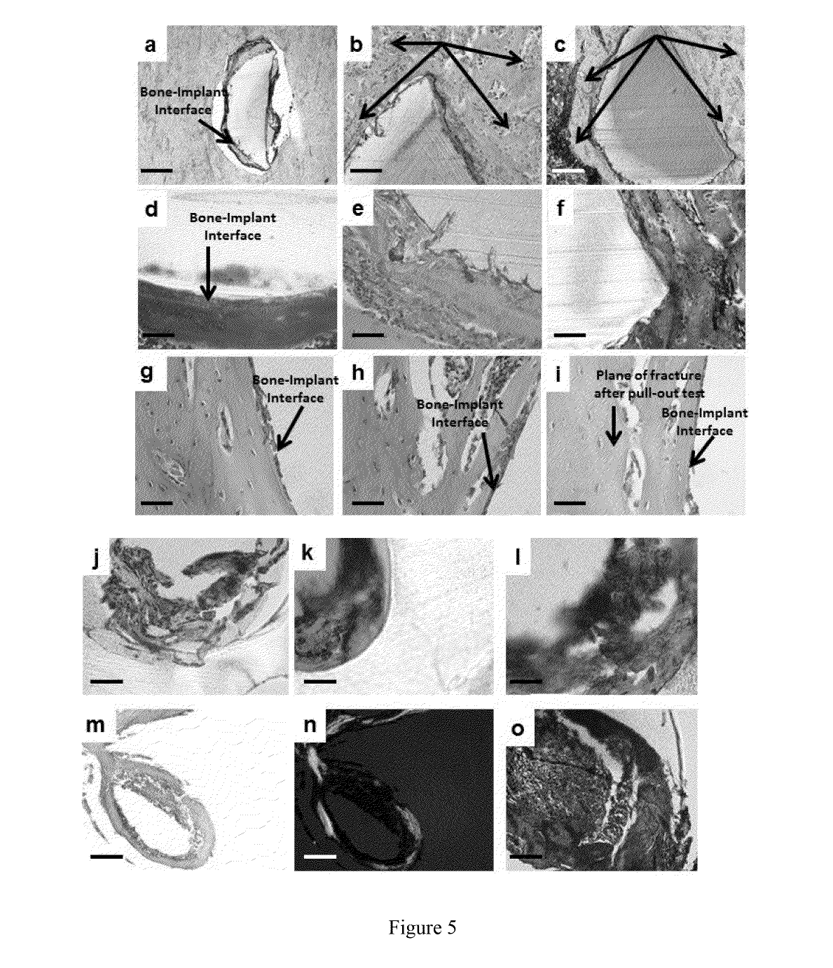

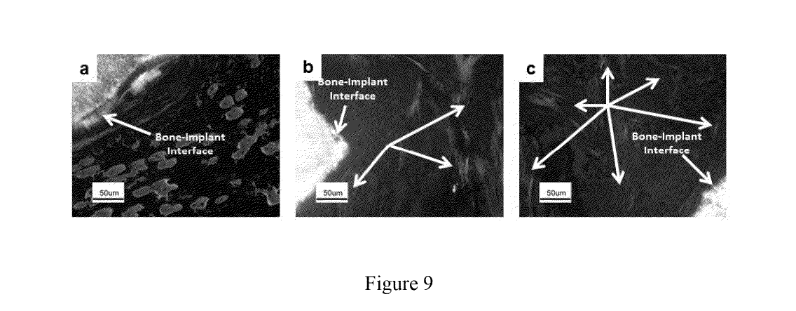

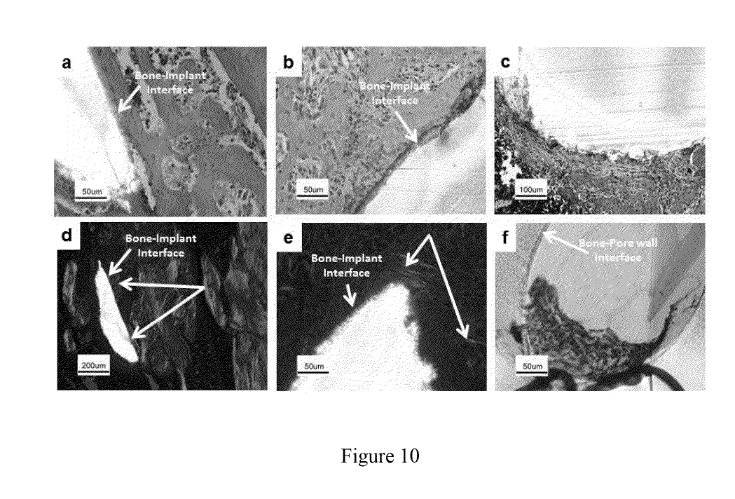

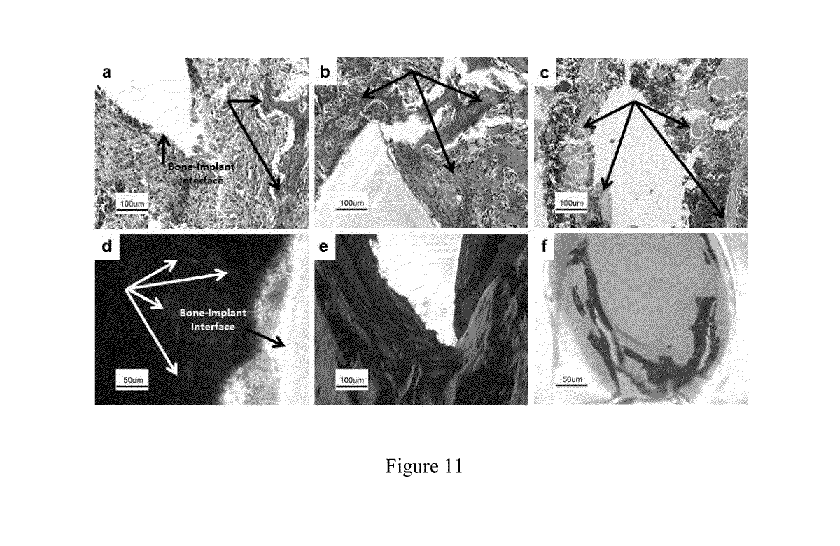

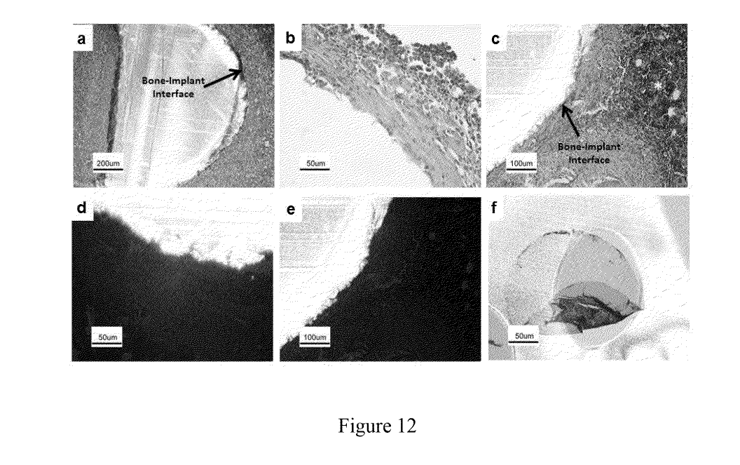

Histological sections of excised tibiae with intact PEEK implants from the rat (FIG. 5) demonstrated active bone formation and remodeling with osteocytes and cement lines (FIG. 5B, 5C, 5E). The new bone is directly laid down on implants containing HAP in the coating by differentiating osteoblasts that reside in the medullary canal without a cartilaginous intermediate (FIG. 5A-F). We observed that having both an osteoconductive and osteoinductive surface promoted trabecular bone deposition on the implant surface, which progressively expanded outward to integrate with the surrounding parent bone (FIG. 5A-C). Consistent with stable bone adhesion, bone tissue was observed on the surface of implants that were pulled out of the tibiae which suggested that the tensile tests resulted in cohesive fracture of bone, rather than failure of the adhesive bone/implant interface (FIG. 5H-I). This finding is significant in light of reports that such failures often occur at the implant interface. Activated, proliferating osteoblasts were observed in the marrow space adjacent to the implants containing rhBMP-2 and newly synthesized bone formation was restricted to the peri-implant space. The collagen fibrils in maturing bone exhibited increasing birefringence under polarized light (FIG. 9) and the mature fibrils oriented along the implant surface and extended outwards, indicating early bone remodeling. A large number of hematopoietic cells were observed around areas of new trabecular bone and the newly synthesized bone was highly vascular, and infiltrated with capillaries. In implants containing drilled holes, maturing bone formed within the implant pores when coated with HAP and rhBMP-2. Calcification was observed along the outer radii with gradual expansion inwards visualized in a loop like conformation in cross section (FIG. 5M, O). This process was not observed in uncoated implants at the time points evaluated in this study, suggesting a role of the synergistic coating in new bone formation on the implant and integration. The filling in of pores with bone is induced by the presence of an HAP coating. Typically, bone infiltrates from the surrounding tissue, without adhesion to the walls of the pores and is thus not as adherent to the implant. The method of direct bone apposition may be highly desirable for one-stage dental implants, where early, direct contact between the bone and implant are important to its success. It is noteworthy that trabecular bone was formed when either HAP or rhBMP-2 were introduced in the multilayer coating. However with HAP alone, bone deposited on the surface of the implant with low contact area (FIG. 10) due to a lack of extensive osteogenic activity and new bone deposition. When rhBMP-2 alone was introduced, the newly synthesized bone did not directly deposit on the implant surface (FIG. 11). Fibrous tissue growth, that is characteristic of a foreign body response, was observed around implants without a coating (FIG. 12), and did not convert into bony tissue over the time course of the study. No osseointegration was observed with untreated drilled or smooth implants. The de novo recapitulation of a more complete bone architecture precipitated by these osteogenic coatings, suggests that there is a synergistic bone regeneration process that can be controlled and harnessed for stable osseointegration.

Rapid, early stabilization of an implant, without the formation of an avascular, loose fibrous tissue capsule are key determinants of long term implant function and integrity. While the unmodified PEEK implant is bioinert, surface mediated introduction of rhBMP-2 and HAP promotes local, synergistic de novo trabecular bone formation that matures over time mechanically and structurally. As a result, the bone generated requires increasing amounts of tensile force to separate from the implant. No bone was observed to form a ‘cap’ around the implant and resistance to tensile forces was entirely due to shear resistance of the adhesive bone. After failure of LbL coated implants, loosely bound, fractured cortical bone was observed on the implant surface. The observation that bone was present on the implant surface after the pull-out test indicates that the shear strength was derived from the new bone and the contributions from the cortical bone were minimal. The uncoated implant was observed to toggle around at the implant site over the course of the study.

Microcomputed tomography (μCT) was used to quantitatively analyze bone formation in the peri-implant space in live animals over time (FIG. 6). From the observations above, interdigitation of the trabecular shell around the implant appears to be occurring at the cortical interface with the endosteal tissue. Calculations were made of the bone volume and the coverage of the bone tissue using a 3D reconstruction. The bone volume in the region of interest was consistently higher when both rhBMP-2 and hydroxyapatite were present in the multilayer coating. Formation of trabecular bone was observed and its thickness generally increased over time around the implant and at the endosteal interface. Ingrowth of bony tissue in drilled implants was evident in 2D slices that increased in density over time. Calcification of the periosteal tissue was observed adjacent to the implant region when the coating contained rhBMP-2. This observation was consistent with the release of rhBMP-2 from the implant and upregulation of osteogenic activity. The deposition of bone on the osteoconductive coating conformal to the implant surface in the medullary canal is evident from the μCT reconstruction. The regeneration of calcified periosteal tissue on the portion of the implant outside the medullary canal was observed very early in the presence of the HAP coating, confirming its utility for direct early bone apposition. The presence of rhBMP-2 resulted in a larger volume of bone in this region. However, the presence of BMP-2 by itself was not sufficient to induce bone apposition to the implant. This result supports the finding that the HAP in the LbL coating retains its osteoconductive property.

In this study we have demonstrated that an efficient and programmable layer-by-layer technique can be used for modifying the surface of implants with tunable hydrolytically degradable coatings that are conformal to the implant surface and induce bone formation directly on the implant surface that is highly adhesive to the implant surface and grows outwards to facilitate implant osseointegration. Our work demonstrates that substrate modification can be used to influence tissue regeneration, confirming the utility of a material-based strategy in regenerative medicine. The coatings described here combine the advantages of synthetic materials with key biologics that regulate tissue development. Each component of the system can be reproducibly synthesized by chemical means with no risk of disease transmission. The coating architecture can be tailored by altering the number of layers and the physical properties of the structural components themselves. The hydroxyapatite and rhBMP-2 retain their osteoconductive and osteoinductive properties respectively and recruit progenitor cells that form new bone in situ in a controlled, localized process that is restricted to the implant surface. The mild, water-based coating scheme is adaptable and very versatile, and in principle several physiologically relevant growth factors for tissue healing applications can be incorporated.

These results provide a unique insight into the cellular mechanism behind the bonding process between an implant surface and bone. The results demonstrate that this coating harnesses the osteogenic potential of precursor bone marrow stem cells by rapidly stabilizing implants in load bearing bone via encapsulation and adhesion with new bony tissue that integrates the implant to the existing bone in a controlled, reproducible process. In the context of bone tissue engineering, it is possible to tune and precisely control cell interactions on the implant surface using synthetic materials to stabilize prostheses and potentially alleviate long term morbidities.

### OTHER EMBODIMENTS AND EQUIVALENTS

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A structure, comprising:
a substrate;
a) a first film disposed on the substrate, which first film comprises at least one bilayer comprised of two polyelectrolyte layers of opposite charges, wherein at least one of the polyelectrolyte layers includes a ceramic material associated with the polyelectrolyte, and
b) a second film comprising at least one tetralayer comprised of four polyelectrolyte layers of alternating first and second opposing charges, wherein:
a first layer, having the first charge, is comprised of at least one hydrolytically degradable polyelectrolyte,
a second layer has the second charge,
a third layer, having the first charge, is comprised of at least one polypeptide; and
a fourth layer has the second charge,
the structure being characterized in that layer-by-layer removal of at least the second film releases at least one polypeptide, and also may permit release of ions from the ceramic material so that a synergetic effect of the osteoinduction and osteoconduction of the structure is achieved.

2. The structure of claim 1, wherein the second film is disposed atop the first film.

3. The structure of claim 2, wherein the second film is disposed immediately adjacent to and atop the first film.

4. The structure of claim 1, wherein the ceramic material is complexed with the at least one polyelectrolyte in the bilayer via a non-covalent interaction.

5. The structure of claim 1, wherein the at least one polyelectrolyte in the bilayer is or comprises a polymer containing amine groups such that it is charged at acidic pH.

6. The structure of claim 1, wherein the at least one polyelectrolyte in the bilayer is or comprises a polysaccharide.

7. The structure of claim 6, wherein the polysaccharide is selected from the group consisting of sodium alginate, chitosan, agar, agarose, carragenaan or any combination thereof.

8. The structure of claim 1, wherein the ceramic material is or comprises a bioceramic, a bioglass or combination thereof.

9. The structure of claim 8, wherein the bioceramic is selected from the group consisting of hydroxyapatite, floroapatite, carbonate apatide, tricalcium phosphate, octacalcium phosphate, calcium pyrophosphate, tetracalcium phosphate, and dicalcium phosphate dehydrate.

10. (canceled)

11. (canceled)

12. The structure of claim 1, wherein the at least one polypeptide in the tetralayer is or comprises a growth factor.

13. The structure of claim 12, wherein the growth factor is osteoinductive.

14. The structure of claim 13, wherein the osteoinductive growth factor is bone morphogenetic protein (BMP).

15-26. (canceled)

27. The structure of claim 1, wherein the substrate comprises at least a portion of a medical device.

28. The structure of claim 1, wherein the substrate comprises at least a portion of an orthopedic implant.

29. The structure of claim 28, wherein the orthopedic implant is or comprises a joint replacement prosthesis.

30. The structure of claim 28, wherein the orthopedic implant is selected from the group consisting of total knee replacement, total hip replacement, ankle replacement, elbow replacement, wrist replacement, and shoulder replacement.

31. The structure of claim 27, wherein the medical device is or comprises a dental implant.

32. (canceled)

33. In an orthopedic implant, the improvement that comprises depositing at least a first and second film on at least a portion of the orthopedic implant,
a) a first film comprising at least one bilayer comprised of two polyelectrolyte layers of opposite charges, wherein at least one of the polyelectrolyte layers includes a ceramic material associated with the polyelectrolyte, and
b) a second film comprising at least one tetralayer comprised of four polyelectrolyte layers of alternating first and second opposing charges, wherein:
a first layer, having the first charge, is comprised of at least one hydrolytically degradable polyelectrolyte,
a second layer has the second charge,
a third layer, having the first charge, is comprised of at least one polypeptide; and
a fourth layer has the second charge,
the coated orthopedicimplant being characterized in that layer-by-layer removal of at least the second film releases the at least one polypeptide, and also may permit release of ions from the ceramic material so that a synergetic effect of the osteoinduction and osteoconduction of the bone implant is achieved.

34. In a method of promoting bone growth on or within an orthopedic implant, the improvement that comprises depositing at least a first and second film on at least a portion of an orthopedic implant,
a) a first film comprising at least one bilayer comprised of two polyelectrolyte layers of opposite charges, wherein at least one of the polyelectrolyte layers includes a ceramic material associated with the polyelectrolyte, and
b) a second film comprising at least one tetralayer comprised of four polyelectrolyte layers of alternating first and second opposing charges, wherein:
a first layer, having the first charge, is comprised of at least one hydrolytically degradable polyelectrolyte,
a second layer has the second charge,
a third layer, having the first charge, is comprised of at least one polypeptide; and
a fourth layer has the second charge,
the coated orthopedic implant being characterized in that layer-by-layer removal of at least the second film releases the at least one polypeptide, and also may permit release of ions from the ceramic material so that a synergetic effect of the osteoinduction and osteoconduction of the bone implant is achieved.

35. The structure of claim 34, a method of making a coated system comprising steps of:
depositing layer-by-layer at least a first and second film onto a substrate,
a) a first film comprising at least one bilayer comprised of two polyelectrolyte layers of opposite charges, wherein at least one of the polyelectrolyte layers includes a ceramic material associated with the polyelectrolyte, and
b) a second film comprising at least one tetralayer comprised of four polyelectrolyte layers of alternating first and second opposing charges, wherein:
a first layer, having the first charge, is comprised of at least one hydrolytically degradable polyelectrolyte,
a second layer has the second charge,
a third layer, having the first charge, is comprised of at least one polypeptide; and
a fourth layer has the second charge,
the coated system being characterized in that layer-by-layer removal of at least the second film releases the at least one polypeptide, and also may permit release of ions from the ceramic material so that a synergetic effect of the osteoinduction and osteoconduction of the system is achieved.

36. A method of using a coated system comprising steps of:
providing a coated system comprising at least a first and second film on a substrate
a) a first film comprising at least one bilayer comprised of two polyelectrolyte layers of opposite charges, wherein at least one of the polyelectrolyte layers includes a ceramic material associated with the polyelectrolyte, and
b) a second film comprising at least one tetralayer comprised of four polyelectrolyte layers of alternating first and second opposing charges, wherein:
a first layer, having the first charge, is comprised of at least one hydrolytically degradable polyelectrolyte,
a second layer has the second charge,
a third layer, having the first charge, is comprised of at least one polypeptide; and
a fourth layer has the second charge,
the coated system being characterized in that layer-by-layer removal of at least the second film; and
releasing the at least one polypeptide and ions from the ceramic material so that a synergetic effect of the osteoinduction and osteoconduction of the system is achieved.

37-40. (canceled)

41. A film comprising at least two polyelectrolyte layers of opposite charges, wherein at least one polyelectrolyte layer comprises a ceramic material associated with the polyelectrolyte so that the ceramic material is stably maintained within the film.

42-51. (canceled)
